SEQUENCE LISTING (1 sequence)

SEQ ID NO: 1 - DNA, 16174 bp, Artificial sequence
  synthetic vector
  misc_feature (1)..(402): PUC57 sequences
  misc_feature (420)..(4142): Tyrosinase Promoter (Mouse) 5′ Section
  misc_feature (4200)..(9452): Tyrosinase Promoter (Mouse) 3′ Section and 5′UTR
  misc_feature (9475)..(9480): Kozak consensus sequence
  misc_feature (9481)..(11376): Leghorn mutant PMEL with anti-miR wobbles coding sequence
  misc_feature (11388)..(11708): miR sequence 1 (anti-native mouse PMEL, in human miR30)
  misc_feature (11709)..(12008): miR sequence 2 (anti-native mouse PMEL, in mouse miR30)
  misc_feature (12009)..(12324): miR sequence 3 (anti-native mouse PMEL, in dog miR30)
  misc_feature (12325)..(12627): miR sequence 4 (anti-native mouse PMEL, in nelore miR30)
  misc_feature (12653)..(13908): SV40 intron, late polyadenylation, and enhancer
  misc_feature (13442)..(13671): SV40 late polyadenylation sequence
  misc_feature (13672)..(13908): SV40 enhancer
  misc_feature (13925)..(16174): PUC57 sequences
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgcggccgccgatcgggcgcccgggctgcaggaattctgtcctaaaacagtctttgggctttctttgggggggcgggggggatttggagtcttttaatgattgcttttattttcttatatattacaggtctatttaaattgttaatatgattttcatttaactttggtaagtgatacctatcaagaaaattgtcatttccaattttatgaagtacaggtttttgacatatgtgctagtaactttctgtgtttccttggtgtctgctgttatgttcccttttggtttcaggttttattgattgggatagtctctcagtgtcttttagttatttttgaataagagtttttctaccttgttgatgttctcaaataacctactctttgtttcattgttaatgtgcattcttttgtttctattttattggttccaggcatgagtttgattatttcctactttttactcctcttgggtgtgtttgtttctttttgtcctagggattttagctgtgctattaagttgttagtatgagatgtctttaatttctttacaaagacacttagtgctaagcacttttctcgtaacaccactttaactgtgtttgggtatatcttgcattccttttcattgaaatctggagagtcttttatttctttctttatttctgccttgacctcaaagtcatccagtagggagttgtgcagctttcatgagtttgtaggttttctgttgttgaatcccacctttactccatgttgtctaataagacacaaggcattatttcaactttcttatatctgttgaggcttattttgtgaacaactatatgatcaattttggagaaagtttaaggattgctgagagaaaggtgccttctttttttgtttgggtgaaatgttctaaaaaatatatattaggtcttttacctccattatttcttagtttagtttttgtctagatgacatgtctattcatgagagtaggatattgaagtctcccactattaatgtattagattcaatttgtgatttgagcattagtaatgtttcacttccaaatgtgttacccttgctttggggacatagatgttaagaactgaagtgttatcatggctaatttttcctttgatgagtatgaaatgtcattttccatctctattgactaattttcttgtaaagtgtgatttgctagatattagaatagctaaaacagcttgcttcttgagtgcatctgcttggaatattttttttcaacccttaactggaggtattgtctatcttgatgttgaggtctattttttgtactcagtgaaatgattgattctctttttgcatccactctgtttgcctgtgtgttttattgagtaattgagtccatttactttgagaaatattaatgatcaatgattcttcatttctgttattttggtggtggtggtgatagtagtgatagttgtgtgcatgtgtatgtgtgtgtgtgtatgtgtgtgtgtttgatttttctctgtatcatgattgcctatatgcaaaactgtaaatatttttcattaatacactagaatttgtgtttaaatgatatacagactaatctataccaaatgtgttattacagattttctagactaaatgactcatcaatcctctgttagatttaaattttgggtaactcttttatatatgtatcctttataatggatttagaagtttgaatcccatcaaaaagacagtacatatatattgtatggaagaatatcaaagttttgtagtgataaaactaggcaattttggcctagagctttgcagtgatgattagtagcaagagtttggttgtagtaatatgttatcacactgtctctctgaaatgatatttctctaaatattcacttctagcaatcatgttcatttgtttacatgaaccaccaaacctaaacatgaactacaaaatgtatgaactttcacccacagtgttattttctcatggctcttgtcctttgttccaaattcaaccagttgaaataatggcagtattttgtaaattctgtacattttttttctctgttccctatacttgacaaaaatttcccttttttcacattcctctatttacactgttcttttttcatggttaagaagagtcagtatgacccttcaagcagtgtttcctaacaatcttgtctcttaggctcatttatagagggaatgagctatgtctccacaatacctcaatattacttgagaataaattccaacatatagacctggtaataagccataagtaccttgttccattgataagtgttcacttggacccatcagttactattccatacatcttcactttctgaacctcatttatgtttgaaaattcaagattagaataactatttaatggaggttatttacaaagatgcattatgtggcatttgtggtaagaatcatttcgtgggtccacaaatccattgcatcattccttgaagtggtttaagtcttttgattgtcattgttaataactgcattaaaagattccttcattccctgatttgagcttgactttactcacagctagaaaattagaggcagaaggagagtgaagtcaaaggtctgcaacttcctctttgccacataacaagaaagttgcttatatccttctccaaggtcatagttcctgccagctgactttgtcaagacagtgatgtctgtgttccagcagttgttctgagtatccttttcattatccactgtcctttcttcttaaattccacccccaacattgtaaatagcttctttcttaaactctgttcaaagaaccagcttgagtgtgtcagctgcttcctgctggggtcctggcaaaccacaatattattaacaatatgtacccaataaagagtcctgaaattgccacaccatcacctttttatattcctattcagattgtgaacttctacaaagcaggaatgtggcttgtttattttcctgttatcttgcactgtcacacttaattcttgggaaaataaaaacacaaaatatgcatttctttggtctactgaatgaagatggaatgtgtattactctgactctttaaatgtaaatggtgcttcatattcctctaaagccttaagcacaataacacagggtgggtggtaagaaaatatctgtagtatggatacaaatttgttccataagaatataagtataaaattattaaataaaatgtttcaggaaaatttaaactggaacaaatcaaggtaagtatcatctaaaaatgagcttaggtagaccatgttgagttgagcttttagggaagctgttaagagtaattatttagaaaaaaagtaggtcaagaagaaaacactaaatagattattcagataaacattttatttcaaatagtattttcatagaacattttgagggcatctgtttggaaagaaattgattattgcattagctctgagattcacatctttcttgtttatataagagctcttttaaaattgtgttttatgatactgtctcttgttgaatgttgcagtctctcctccaattcagacatctccaagggtcaggaagcaacatgagcaattcagtgaaagatagtatatttccaatgtctaaccaaaaattgtagattctgatgaaagcctggttgtgtgatgagacacacagagagagaatcagacttggaaaatggctgcacagctacaaatacttcaaatacttgctatcaagcttaacggcatgaattcgatatcaagcttatcgataccgtcgactaatacgactcactatagggcgagaattcggatcaatatagtaaaaatggctatcttgccaaaagcaatctacagattcaatgaaatccccatcaaaattccaactcaattcttcaacgatttagaaagggcaatctgcaaattcatctggaataacaaaaaacctaggatagcaaaaagtcttctcaaggataaaagaacctctggtggaatcaccatccctgacctaaagctgtactacagagcaattgtgataaaaactgcatggtactggtatagcaacagacaagtagaccaatggaatagaattgaagacccagaaatgaacccacacacctatggccatttgatctttgacaagggagctaaaaccatccagtggaaaaaagacagcattttcaacaaatggtgctggcacaactggtggttatcatgtagaagaatgggaattgatccattcctatctccttgtactaaggtcaaatctaagtggatcaaggaaccccacataaaagcagagacagtgtaacttatagaggagaaagtggggaaaagccttgaagatatgggcacatgggaaagattcctgaatagaacagcaatgacttgtgctgtaagattgagaatcagccaatgggacctcataaaattgcaaagcttctgtaaggcaaaagacaccatcataagacaaataggccaccaacagattgggaaaggatctttacctatcctaaatcagataggggactaatatctaatatatataaagaactcaagaaggtggactccagaaaatcaaataactccattaaaaaatggggctcagagctaaacaaagaattctcacctgaggaataccaaatggctgagaagcacctgaaaaaatgtttaacatccttaatcatcagggaaatgcaaattaaaacaaccctgagattccatctcacatcagtcagaatggctaagatcaaaatttcaggtgacagcagatgctggcaaggatgtgaagaaagaggagcactcctccatttttggtgagtttgcaagcttgtacaaccactctggaaatcagtctggtggttactcaaaaaatttgacacagtactgctggaggatccagcaatacctctcctggggcatatatttagaagatgttccaaccagtaagaaggacacatgctccactatgttcatagcagccttatttataatagccagaagctgggaagaacccctcaacagaggaatggatacagaaaatgtggtacatttacacaatggagtactactcagctattaaaaagaatgaatttatgaaatttctaggcaaatggatgggcctgcagggcatcatcttgagtgaggtaactcaatcacaaaagaactcacatgatatgtactcactaataagtggaagagaggtttcttggtcttgcaaactttatatgccccagttcaggggaacaccagggccaagaaatgggagtgagtggataggggagcaggaccgcatggtggtggggtgggggggtgggtatagggaactttcaggatagcatttgaaatgtaaatgaagaaaatatctaataaaattttttaaaaaaccttaaaaaagagagagaaaaagaactgtaggagctgcatatttacagaataaattagtatgtctaaactccaaatatagagaattaatcactgtaaactttttcctttagttgtatctaagcacacaagtaagcaaaactatagtggtagttaacatttgtatctgaaattgacagatggctcagcagaaaaaggcacttactctcaagcctgaagacaggagtttaatcaacaaatccgcaggctgaagaaaagaattgactctttcaagttgtcttctaatttccactgaggtatcaggcacacattgttcatgtatatacacatagctacaaagaaacacacaaatacacataacacacacacacacacaaatttaccacatgcccaaactaataactaattgtaataaaaaatacgtatcatatccagtgataattcacctcaatgcaaaatgaaatcatgtggaatcatttttagtaaatgtaagacaatgacagaattaggaagccagtcattcttgcaatatttagcaaaacaatgtatataaaaagccattcttgaaaataataaaaaccttacaaatttttggtagaaaattatatgacagaatagtctgaaatctcttcaagctgggaaaacctaaagctactgcactcaagtatcctggaataatgagtactgtcctaattttctttcttgctgtgattaaacaattctgtctcaaaaacaacctaaggaagaaaaatttatactggctagcagttccccatcctgatataaaagtcagggcaggatgggactgaagtacctaccccacatataaaactagaatgtacaaaggaaggaattcatgctcgtgttcagtttgctttcttcattttttgaagtctacaatctcatgtctagggaatgatcccattacaattgagatgagtctctcgacaccagtcatagtcaagtgaatgcccacagtatgcccagaagaccatctcccaagtgattctggattctatcaagttgacaaaaaactatcacaagttgaaaaaaaaaaaaaaaaggacttttcttcataagatacagagagaatggaacacatgaataatgtaatgctagggaaacagatgcaaagagttcaggaattcatgccccagttgacaacatagctggttcaagacaaacctgagcacttttcatcactgaaatcttcactctggaccaatcaacattcatacattcccttctttactttaacactctcctgagagctatttctcttctcatcctaattctctgctcatatcacatttcagcagcttacatatgaaaatcttgtacattcccatgagattgcattgaaattgcttcaacccttttctatgtccatatgtataccttacttctcattcctattgctttgtagctacagaagccttagcagtctttagaaatcttgggagtgttatgcctcttccacttaaagctttaattcaagaactcattctctagaagtttaaacaactgctattctgctcttctatgactctttaacatgtctctcaaaatatgttttctcccagaaaactctcctcaactttcttcaaaattaatagactacattttgccccacagaaattcttctataggaacttcaatttttttatttaatccaggaaaacatatttaaacatacttagctataaatattaagattaaattgtgataaaaacttatttttgacagtggtggaaactgtcctattaaatttaccatatcattccaattttatcctcagaaaatgcccaatagatgatcaataaatatttgcagtatacttattgcattcccagctatagctaatatagattgcattaagactgtcttagggtagacaatagtcaaacagaattggatagggcatattcaaattctgtagctaccaaatgtgtatacaagggaaaaatgtgtaaatgaatacttacatagaaagagcaggactggtgataagattgtcatgtaaataagttggtggagactgtagattacattgcagaggaataaagagaacttttaatttaagagaatcagtcctatgaagaaaacaaagacagcaaactacacttcagtcattcctctcatgaggctctacagattttgaaaatgagcatgggaaagcccttagaaaatgagcatgggaaagcccttagaaagagtgatgaggattttctgagcaattttgctaagatgcctgatagtagaacaagggaaagaaaaagttgttaattaagtttaacaggagaagctggattccacctgcaactaggtgggaaagagttacaaaaggccccatgtagctacaagtaatacataacaaaaagtccccaggggaacgaggtcctgtgttaatctactatgggctttaagagaagaaaaacaaaaaaggactaagactcctgaaggaaatcatgtgagagcttccttattccagcaagagacaaaatctccatggaaacttctctttcctgcacccacacactgttctttctacctgccaaggctgccttgaatctcaagagaatcctatggagcagtcaacacattttaaatactgaaacaaaccctagggaagaaaagaagcagagcaggctgacattccagcattatcaggaaagcaatgattttcctagatttccgcagccccagtgttcagataaacggtttcctcaaaccttcacttcctttctcctacagtataaattaaaagaaccactcagcttttagtatgaagcagcatagagaagggagttccaaagagacgtttgtttcttgaccattctcattatccttctttcatggagcagtgctattcaaaccatccagtaagtccattactcacttccacattttatgagcaaaataataaaagagagatagagtaagaatgaaggagagagagagagagagagagagagagagagagagagagagagagagagagaggaagagagagagagagagagagagagagagagagagagagagagagagagagagagagagagaggtgttttcttaactagaaactttatgcattgaagcagttcaccaaaataacaaagtaacaaagtaagatatctttggaataatcaattcaagataatcaaggaaaaatgagaggcaactattttagactgattacttttataaaataaataagctcagcttagccagatataagcaatattctgagttctgaagaaaaatttttgacaaaatgagttctataaatgttattgtctacttatgatctctaaatacaacaggcttgtattcagaatctagatgtttcatgacctttattcataagagatgatgtattcttgatactacttctcatttgcaaattccaattattattaatttcatatcaattagaataatatatcttccttcaatttagttacctcactatgggctatgtacaaactccaagaaaaagttagtcatgtgctttgcagaagataaaagcttagtgtaaaacaggctgagagtatttgatgtaagaaggggagtggttatataggtcttagccaaaacatgtgatagtcactccaggggttgctggaaaagaagtctgtgacactcattaacctattggtgcagattttgtatgatctcctaggcactagtctcgagtgcacgtacgccaccatgggtgtccagagaaggagcttccttcccgtgcttgtgctgagtgctctgctggctgtgggggccctagaaggttccaggaatcaggactggcttggtgtaccgaggcagctagtcaccaaaacctggaacaggcagctgtaccccgagtggacagaggtgcaggggtctaactgctggagaggtggccaggtatctctgagggtcattaatgatgggcctacactggttggtgcaaatgcctccttttccattgccctgcacttccctggaagtcaaaaggtactaccggatggtcaggttatctgggccaacaacaccatcatcaatgggagccaggtgtggggaggacagccagtgtatccacaggagcctgatgatgcctgtgtcttccctgacggtggaccctgcccatctggtcctaaaccacctaaaaggagtttcgtctatgtttggaagacctggggaaaatactggcaagttctggggggtccagtgtccaggctgagcattgctacgggccacgcaaagctgggcacacacacaatggaagtgactgtctaccaccgacggggttcccagagctacgtgccccttgctcacgcgagctctacgtttaccatcactgaccaggtacctttctccgtgagtgtgtcccagctacaggccttggacggagagaccaagcacttcctgagaaatcatcctctcatctttgcccttcagctccacgaccccagtggttatttggccgaggccgacctctcctacacatgggactttggagatggtactgggaccctgatctctcgggcacttgatgtcactcacacttacctggagtcgggctcagtcactgcccaggtggtactgcaggctgccattcctcttgtttcctgtggttcctccccagttccgggtaccacagatggctacatgccaactgcagaagcacctggaaccacatctaggcaaggaaccactacaaaagttgtgggtactacacctggccagatgccaactacacagccctctggaaccacagttgtacaaatgccaaccacagaggtcactgcaaccacttcggaacaaatgctgacctcagcggtcatagataccacactggcagaggtgtcaactacagagggtacaggtaccacacccacaaggccttctggaaccaccgttgcacaagcaacaaccacagagggtccagatgccagcccattgctgcccacacaaagttctacagggtccattagccctctactggatgacaccgacaccataatgcttgtgaagagacaagttcccctggactgtgttctatatcgatatggttctttctccctcgccctggacattgtccagggtattgaaagtgctgagatcctgcaggctgtgccattcagtgaaggggatgcatttgagctgactgtgtcctgccaaggcgggctacccaaggaagcctgtatggacatttcatcaccagggtgccagccccctgcccagaggctgtgccagtctgttccaccgagcccagactgccagctggttctacaccaagtgctgaaaggtggctcagggacatattgcctcaatgtgtctttggctgacgccaacagcctggcagtggccagcacccaacttgttgttcctggtcaagacggtggccttgggcaggctcccttgcttgtaggtatcttgtgggctccactggtgttggtggctgtggtccttgcatctctgatacataggcatagacttaagaagcagggctcagtttcccaaatgccacatggtagcactcactggctgcgcctgcctccggtcttccgcgcccgcggccttggagaaaacagcccgctcctcagtggacagcaggtctgattcgaacgcgttgtttgaatgaggcttcagtactttacagaatcgttgcctgcacatcttggaaacacttgctgggattacttcttcaggttaacccaacagaaggctaaagaaggtatattgctgttgacagtgagcgatcccaagacaacttgtaactatagtgaagccacagatgtatagttacaagttgtcttgggactgcctactgcctcggacttcaaggggctactttaggagcaattatcttgtttactaaaactgaataccttgctatctctttgatacatttttacaaagctgaattaaaatggtataaattaaatcactttagggcatcgaggctttgcagtttacagaatgttgcctgtacatttcagaaactcccgcagagcacttctcaggtcagtaagacagaatgctgaagaaggcatatcaacgttgacagtgagcgatccgaagagaagctttgtttatagtgaagccacagatgtataaacaaagcttctcttcggagtgcctactgcctccaacttcaaggagtaattatcttgttggctgaaaccaataccttgctttctctttgatttatttttacaaagcttgggtggaaaagataaattaaacaactttgttagagtgagaatttggtaacgtacagaattgctgtttgtacatctcagaaacacctgctagggaccagtctgcaggttaaccagacagaaagaaggtatatccctcttgacagtgagcgaccagttcaaccttcaccattatagtgaagccacagatgtataatggtgaaggttgaactggctgcctactgcctcagacttcaaggagctactttaggtgcaattatcttgttaactaaaactgaataccttgccttctctttgattcatttctacaaagcttgggtgaaaatgatatgaattaaatgacttttttctaggtttgggagaagacttaatggtgtgcagaattgctgcctgtctgtcttagggaccctcaggtaaccagacagcaaggagtgtccctgtgacagtgagcgccagctactacatctgagcagatagtgaagccacagatgtatctgctcagatgtagtagctgttgccgactgccgcccacgtcaggagcccccttaggagcagtggcctccttgactccactggacactgtgctctcctgggaatacgtgttcacatccttgcgtgagaatggtacaggttcagtaacttttaaaccgacgtcgcgcgcccctagggctagaaatgtaactgtattcagcgatgacgaaattcttagctattgtaatactgcgatgagtggcagggcggggcgtaatttttttaaggcagttattggtgcccttaaacgcctggttgctacgcctgaataagtgataataagcggatgaatggcagaaattcgccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgaaccatggagcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagcgtcgacagatctggccggccgcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2019/0300901 A1
(43) Pub. Date: Oct. 3, 2019

(54) MATERIALS AND METHODS FOR MAKING A RECESSIVE GENE DOMINANT

(71) Applicant: AgGenetics, Inc., Nashville, TN (US)

(72) Inventor: James West, Nashville, TN (US)

(73) Assignee: AgGenetics, Inc., Nashville, TN (US)

(21) Appl. No.: 16/290,584

(22) Filed: Mar. 1, 2019

Related U.S. Application Data
(63) Continuation of application No. 14/536,603, filed on Nov. 8, 2014, now abandoned.
(60) Provisional application No. 61/902,176, filed on Nov. 9, 2013.

(51) Int. Cl. C12N 15/85; C12N 15/113; A01K 67/027; C12N 15/877; C12N 15/87

(52) U.S. Cl. CPC C12N 15/8509; C12N 15/1138; A01K 67/0275; C12N 15/8771; C12N 15/87; A61K 48/0066; C12N 2830/20; A01K 2267/02; A01K 2217/206; A01K 2217/072; A01K 2217/058; C12N 15/85

(57) ABSTRACT

The subject invention provides materials and method for making a recessive gene dominant. This is accomplished by interfering with the natural mechanisms that inhibit expression of the recessive gene and/or by interfering with the expression of the naturally dominant gene. In a preferred embodiment, the method of the subject invention comprises both reducing inhibition of expression of the recessive gene and increasing inhibition of the dominant gene.

### CROSS-REFERENCE TO RELATED APPLICATIONS

This is a Continuation of U.S. patent application Ser. No. 14/536,603, filed Nov. 8, 2014, which claims the priority benefit of U.S. Provisional Application Ser. No. 61/902,176, filed Nov. 9, 2013, each of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 186122000501SEQLIST.TXT, date recorded: Feb. 28, 2019, size: 22 KB).

### BACKGROUND OF THE INVENTION

A recessive gene is an allele that results a phenotype that is only seen in a homozygous genotype (an organism that has two copies of the same allele) and never in a heterozygous genotype. Thus, if a genetic trait is recessive, the animal needs to inherit two copies of the gene for the trait to be expressed.

Micro RNAs (miRNAs) are small non-coding RNA molecules found in plants and animals, which function in transcriptional and post-transcriptional regulation of gene expression. They consist of short sense and antisense sequences (˜24 base pairs each) with close sequence similarity to their targets. When they are transcribed as single stranded RNAs, they make hairpin loops, and are processed by the RNA-induced silencing complex (RISC) into the functional microRNAs. Encoded by eukaryotic nuclear DNA, miRNAs function via base-pairing with complementary sequences within mRNA molecules, usually resulting in gene silencing via translational repression or target degradation.

Animal miRNAs typically exhibit only partial complementarity to their mRNA targets. A ‘seed’ region of about 6-8 nucleotides in length at the 5′ end of an animal miRNA is thought to be an important determinant of target specificity.

### BRIEF SUMMARY

The subject invention provides materials and method for making a recessive gene dominant. This is accomplished by interfering with the natural mechanisms that inhibit expression of the recessive gene and/or by interfering with the expression of the naturally dominant gene. In a preferred embodiment, the method of the subject invention comprises both reducing inhibition of expression of the recessive gene and increasing inhibition of the dominant gene.

In embodiments specifically exemplified herein, the natural inhibition of the recessive gene is reduced by changing the sequence of the recessive gene such that miRNA that would normally inhibit the expression of the gene no longer binds to the recessive mRNA and, thus, does not inhibit the expression of the gene.

In preferred embodiments, the changes to the polynucleotide encoding the recessive gene do not result in a change to the amino acid sequence of the encoded protein or, if there is a change, it is a minor change that does not adversely affect the functionality of the protein. Such changes in sequence can be achieved via, for example, taking advantage of the degeneracy of the genetic code and the associated third base “wobble.” In another embodiment, the gene for a recessive gene in one species (the target species) can be replaced with a gene encoding the same protein in another species in which the native gene already has a significant amount of mismatch to the miRNA in the target species.

In a further embodiment of the present invention, the expression of the dominant gene is inhibited by the introduction of miRNA that targets the RNA for the protein expressed by the dominant gene. In a preferred embodiment, multiple miRNAs to the same gene are incorporated into the 3′ untranslated region (UTR) thereby significantly enhancing the knockdown of the formerly dominant gene. Thus, in one embodiment of the present invention, multiple miRNAs that target a single dominant gene are provided in polycistronic strings.

The present invention also provides expression constructs and vectors for making recessive genes dominant.

The subject invention further provides animals produced according to the methods of the subject invention.

### BRIEF DESCRIPTION OF THE FIGURE

FIG. 1 shows a plasmid vector for a construct for use according to the method of the subject invention.

### BRIEF DESCRIPTION OF THE SEQUENCE

SEQ ID NO:1 shows a plasmid vector for a construct for use according to the method of the subject invention.

### DETAILED DISCLOSURE

The current invention provides materials and methods for customizing animal traits, wherein the invention utilizes knowledge of the genetic nature of a trait in an animal and targeted gene modification. In one embodiment, the methods of the subject invention utilize spermatogonial stem cell (SSC) transfer to allow production of trait-customized sperm. In other embodiments, the methods of the subject invention can utilize somatic-cell nuclear transfer (SCNT).

The customization of traits is achieved according to the subject invention via a method for making a recessive gene dominant. This is accomplished by interfering with the natural mechanisms that inhibit expression of the recessive gene and/or by interfering with the expression of the naturally dominant gene. In a preferred embodiment, the method of the subject invention comprises both reducing inhibition of expression of the recessive gene and increasing inhibition of the dominant gene.

In embodiments specifically exemplified herein, the natural inhibition of the recessive gene is reduced by altering the sequence of the recessive gene such that miRNA that would normally inhibit the expression of that gene no longer binds to the mRNA of the recessive gene and, thus, does not inhibit the expression of the gene.

In preferred embodiments, the changes to the polynucleotide encoding the recessive gene do not result in a change to the amino acid sequence of the encoded protein or, if there is a change, it is a minor change that does not adversely affect the functionality of the protein. Such changes in sequence can be achieved via, for example, taking advantage of the degeneracy of the genetic code and the associated third base “wobble.” In another embodiment, the gene for a recessive gene in one species (the target species) can be replaced with a gene encoding the same protein in another species in which the native gene already has a significant amount of mismatch to the miRNA in the target species.

In a further embodiment of the present invention, the expression of the dominant gene is inhibited by the introduction of miRNA that targets the RNA for the protein expressed by the dominant gene. In a preferred embodiment, multiple miRNAs to the same gene are incorporated into the 3′ untranslated region (UTR) thereby significantly enhancing the knockdown of the formerly dominant gene. Thus, in one embodiment of the present invention, multiple miRNAs that target a single dominant gene are provided in polycistronic strings.

In one embodiment, the present invention provides a method for reducing the dominance of a naturally dominant nucleic acid sequence in an animal and increasing the dominance of a naturally-recessive nucleic acid sequence, wherein the method comprises:

obtaining one or more spermatogonial stem cells (SSCs) of a male animal that has a dominantly acting endogenous nucleic acid molecule;

providing a modification construct comprising an exogenous polycistronic inhibitory RNA nucleic acid sequence that suppresses the dominantly acting endogenous nucleic acid molecule, and further providing an exogenous nucleic acid sequence of the recessively acting nucleic acid molecule in which base mutations in at least one codon have been introduced or exist (compared to the wild-type sequence in that species) to prevent or reduce binding of inhibitory RNA molecules; and

introducing the modification construct(s) into at least one of the SSCs, thereby obtaining at least one SSC comprising a nucleic acid molecule that suppresses the dominantly acting endogenous nucleic acid molecule and a second nucleic acid molecule that expresses a previously recessively acting nucleic acid molecule having a different sequence than the wild-type polynucleotide that expresses the naturally recessive gene; and

introducing one or more modified SSCs into a reproductive organ of a male recipient animal; and optionally,

collecting the donor-derived, fertilization-competent, haploid male gametes produced by the male recipient.

The corrective methods of the subject invention can also be practiced using somatic-cell nuclear transfer (SCNT). Any somatic cell including, for example, skin fibroblasts, can be isolated from the target animal. The recessive mutation is corrected in that cell, by the same methods exemplified herein for SSC. Well-known somatic cell nuclear transfer technologies (cloning) can then be used to create an animal genetically identical to the target animal, but with recessive mutations corrected.

In a preferred embodiment the modification construct comprises a nucleic acid sequence encoding a polycistronic inhibitory RNA molecule, wherein the polycistronic RNA molecule comprises multiple inhibitory RNA molecules, wherein the inhibitory RNA molecules suppress a dominantly acting endogenous nucleic acid sequence. In one embodiment, the modification construct comprises an exogenous nucleic acid sequence of the recessively acting nucleic acid molecule in which base mutations in at least one codon have been introduced to prevent, or reduce, binding of inhibitory RNA molecules.

In one embodiment the nucleic acid sequence encoding the polycistronic inhibitory RNA molecule and the nucleic acid sequence encoding a mutant, miRNA-resistant version of the recessively acting nucleic acid are present on the construct.

In one embodiment the nucleic acid sequence encoding a polycistronic inhibitory RNA molecule and the nucleic acid sequence encoding a mutant, inhibitory RNA-resistant version of the recessively acting nucleic acid are present on different constructs.

In one embodiment, the genome of at least one modified SSC comprises a nucleic acid molecule comprising a nucleic acid sequence encoding a polycistronic inhibitory RNA molecule and a nucleic acid sequence encoding a mutant, inhibitory RNA-resistant version of the recessively acting nucleic acid molecule.

### Definitions

As used herein, “Angus” refers to any bovine animal with any Angus ancestry.

The term “recessive allele,” as used herein, refers to its ordinary meaning that is an allele whose phenotype is not expressed in a heterozygote.

The term “dominant allele,” as used herein, refers to its ordinary meaning that is an allele whose phenotype is expressed in a heterozygote.

As used herein, the term “expression construct” refers to a combination of nucleic acid sequences that provides for transcription of an operably linked nucleic acid sequence. Expression constructs of the invention also generally include regulatory elements that are functional in the intended host cell in which the expression construct is to be expressed. Regulatory elements include promoters, transcription termination sequences, translation termination sequences, enhancers, and polyadenylation elements.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

As used herein, the term “operably linked” refers to a juxtaposition of the components described wherein the components are in a relationship that permits them to function in their intended manner. In general, operably linked components are in contiguous relation. Sequence(s) operably-linked to a coding sequence may be capable of effecting the replication, transcription and/or translation of the coding sequence. For example, a coding sequence is operably-linked to a promoter when the promoter is capable of directing transcription of that coding sequence.

A “coding sequence” or “coding region” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus.

The term “promoter,” as used herein, refers to a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

In addition to a promoter, one or more enhancer sequences may be included such as, but not limited to, cytomegalovirus (CMV) early enhancer element and an SV40 enhancer element. Additional included sequences are an intron sequence such as the beta globin intron or a generic intron, a transcription termination sequence, and an mRNA polyadenylation (pA) sequence such as, but not limited to, SV40-pA, beta-globin-pA, the human growth hormone (hGH) pA and SCF-pA.

In one embodiment, the expression construct comprises polyadenylation sequences, such as polyadenylation sequences derived from bovine growth hormone (BGH) and SV40.

The term “polyA” or “p(A)” or “pA” refers to nucleic acid sequences that signal for transcription termination and mRNA polyadenylation. The polyA sequence is characterized by the hexanucleotide motif AAUAAA. Commonly used polyadenylation signals are the SV40 pA, the human growth hormone (hGH) pA, the beta-actin pA, and beta-globin pA. The sequences can range in length from 32 to 450 bp. Multiple pA signals may be used.

The term “vector” is used to refer to any molecule (e.g., nucleic acid, plasmid, or virus) used to transfer coding information (e.g., a polynucleotide of the invention) to a host cell.

The terms “expression vector” and “transcription vector” are used interchangeably to refer to a vector that is suitable for use in a host cell (e.g., a subject's cell) and contains nucleic acid sequences that direct and/or control the expression of exogenous nucleic acid sequences.

Expression includes, but is not limited to, processes such as transcription, translation, and RNA splicing, if introns are present. Vectors useful according to the present invention include plasmids, viruses, BACs, YACs, and the like. Particular viral vectors illustratively include those derived from adenovirus, adeno-associated virus and lentivirus.

The term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

The term “recombinant” is used to indicate a nucleic acid construct in which two or more nucleic acids are linked and which are not found linked in nature.

The term “nucleic acid” as used herein refers to RNA or DNA molecules having more than one nucleotide in any form including single-stranded, double-stranded, oligonucleotide or polynucleotide.

The term “nucleotide sequence” is used to refer to the ordering of nucleotides in an oligonucleotide or polynucleotide in a single-stranded form of nucleic acid.

The term “expressed” refers to transcription of a nucleic acid sequence to produce a corresponding mRNA and/or translation of the mRNA to produce the corresponding protein.

Expression constructs can be generated recombinantly or synthetically or by DNA synthesis using well-known methodology.

The term “regulatory element” as used herein refers to a nucleotide sequence which controls some aspect of the expression of an operably linked nucleic acid sequence. Exemplary regulatory elements illustratively include an enhancer, an internal ribosome entry site (IRES), an intron, an origin of replication, a polyadenylation signal (pA), a promoter, a transcription termination sequence, and an upstream regulatory domain, which contribute to the replication, transcription, post-transcriptional processing of a nucleic acid sequence. Those of ordinary skill in the art are capable of selecting and using these and other regulatory elements in an expression construct with no more than routine experimentation.

In one embodiment, the construct of the present invention comprises an internal ribosome entry site (IRES). In one embodiment, the expression construct comprises kozak consensus sequences.

A “gene” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a coding sequence for any polypeptide or fragment thereof, a functioning version of a malfunctioning endogenous molecule or a malfunctioning version of a normally-functioning endogenous molecule. An exogenous molecule can also be the same type of molecule as an endogenous molecule but be derived from a different species than the species the endogenous molecule is derived from. For example, a human nucleic acid sequence may be introduced into a cell line originating from a hamster or mouse.

An “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP DNA-binding domain and a cleavage domain) and fusion nucleic acids (for example, a nucleic acid encoding the fusion protein described supra).

“Complement” or “complementary sequence” means a sequence of nucleotides which forms a hydrogen-bonded duplex with another sequence of nucleotides according to Watson-Crick base-pairing rules. For example, the complementary base sequence for 5′-AAGGCT-3′ is 3′-TTCCGA-5′. This invention encompasses complementary sequences to any of the nucleotide sequences claimed in this invention.

### Construct Design and Delivery

In one embodiment, the modification construct further comprises an excisable selection marker. Examples of selection markers useful according to the present invention include, but are not limited to, antibiotic resistance, fluorescent cell sorting marker, magnetic cell sorting marker, and any combination thereof. Suitable selection marker genes are known in the art, including but not limited to, nucleic acid molecules encoding proteins that mediate antibiotic resistance (e.g., ampicillin resistance, neomycin resistance, G418 resistance, and puromycin resistance), nucleic acid molecules encoding colored or fluorescent or luminescent proteins (e.g., green fluorescent protein, enhanced green fluorescent protein, red fluorescent protein, and luciferase), and nucleic acid molecules encoding proteins that mediate enhanced cell growth and/or gene amplification (e.g., dihydrofolate reductase). Epitope tags include, for example, one or more copies of FLAG, His, myc, Tap, HA or any detectable amino acid sequence.

The selection marker can be excisable by any recombinase (e.g., Piggyback™, Cre-Loxp recombinase, and Flp recombinase). Vector designs of Piggyback™, Cre-Loxp recombinase, Flp recombinase for excision of nucleic acid sequences are known in the art.

If desired, the vector may optionally contain flanking nucleic sequences that direct site-specific homologous recombination. The use of flanking DNA sequences to permit homologous recombination into a desired genetic locus is known in the art. At present it is preferred that up to several kilobases or more of flanking DNA corresponding to the chromosomal insertion site be present in the vector on both sides of the encoding sequence (or any other sequence of this invention to be inserted into a chromosomal location by homologous recombination) to assure precise replacement of chromosomal sequences with the exogenous DNA. See e.g. Deng et al, 1993, Mol. Cell. Biol 13(4):2134-40; Deng et al, 1992, Mol Cell Biol 12(8):3365-71; and Thomas et al, 1992, Mol Cell Biol 12(7):2919-23. It should also be noted that the cell of this invention may contain multiple copies of the gene of interest.

In one embodiment, the modification construct is introduced into the SSCs using a site-specific nuclease. Site-specific nucleases useful according to the present invention include, but are not limited to, transcription activator-like effector nucleases (TALENs), zinc-finger nucleases (ZFNs), and/or clustered regulatory interspaced short palindromic repeat (CRISPR)/Cas-based RNA-guided DNA endonucleases. TAL-effector nucleases are a class of nucleases that allow sequence-specific DNA cleavage, making it possible to perform site-specific gene editing.

Site-specific genome-editing materials and methods are known in the art. In certain embodiments, a site-specific nuclease is introduced to the host cell that is capable of causing a double-strand break near or within a genomic target site, which greatly increases the frequency of homologous recombination at or near the cleavage site. In certain embodiments, the recognition sequence for the nuclease is present in the host cell genome only at the target site, thereby minimizing any off-target genomic binding and cleavage by the nuclease.

In one embodiment, the site-specific nuclease recognizes a target sequence. In one embodiment, the site-specific nuclease is engineered to cleave a pre-determined nucleic acid sequence from the endogenous nucleic acid molecule, wherein the pre-determined sequence is located near the endogenous dominantly acting nucleic acid sequence.

Site-specific nucleases can be introduced into the SSCs using any method known in the art. In one embodiment, the site-specific nuclease enzymes are introduced directly into SSCs. In another embodiment, the present invention involves administering a nucleic acid molecule encoding a site-specific nuclease into the SSCs. In one embodiment, the nucleic acid molecule encoding the SSCs is in an expression vector. In one embodiment, the correction vector comprises a nucleic acid molecule encoding a site-specific nuclease.

The site-specific nuclease can be introduced into the SSCs before, during (or simultaneously), and/or after the administration of the correction vector to the SSCs.

### Target Animals

The animals whose recessively acting nucleic acid sequence(s) can be made dominant in accordance with the present invention can be of any species, including, but not limited to, mammalian species including, but not limited to, domesticated and laboratory animals such as dogs, cats, mice, rats, guinea pigs, and hamsters; livestock such as horses, cattle, pigs, sheep, goats, ducks, geese, and chickens; primates such as apes, chimpanzees, orangutans, humans, and monkeys; fish; amphibians such as frogs and salamanders; reptiles such as snakes and lizards; and other animals such as fox, weasels, rabbits, mink, beavers, ermines, otters, sable, seals, coyotes, chinchillas, deer, muskrats, and possum.

In certain embodiments, the animals are from any family of Equidae, Bovidae, Canidae, Felidae, and Suidae. In one embodiment, the animal is not a human. In one specific embodiment, the animal is a bovine animal. In a preferred embodiment, the bovine animal is of the black Angus breed. In certain embodiments, bovine animals of the present invention can include, but are not limited to, domesticated cattle, bison, and buffalos (e.g., water buffalo and African buffalo).

### Nuclease-Mediated Site-Specific Genome Editing

Methods of site-specific genome editing are known in the art. In certain embodiments, the present invention uses transcription activator-like effector nucleases (TALENs), zinc-finger nucleases (ZFNs), and/or clustered regulatory interspaced short palindromic repeat (CRISPR)/Cas-based RNA-guided DNA endonucleases for site-specific genome editing, all of which are known in the art. See Gaj et al., ZFN, TALEN, and CRISPR/Cas-Based Methods for Genome Engineering, Trends in Biotechnology, July 2013, Vol. 31, No. 7, which is hereby incorporated by reference in its entireties.

TALENs (transcription activator-like effector nucleases) are fusions of the nuclease (such as FokI) cleavage domain and DNA-binding domains derived from TALE proteins. TALEs contain multiple 33-35-amino-acid repeat domains that each recognizes a single base pair. TALENs can induce double-strand breaks that activate DNA damage response pathways and enable custom alteration.

ZFNs (zinc-finger nucleases) are fusions of the nonspecific DNA cleavage domain from a restriction endonuclease (such as FokI) with zinc-finger proteins. ZFN dimers induce target DNA double-strand breaks that stimulate DNA damage response pathways. The binding specificity of the designed zinc-finger domain directs the ZFN to a specific genomic site. ZFNickases (zinc-finger nickases) are ZFNs that contain inactivating mutations in one of the two nuclease (such as FokI) cleavage domains. ZFNickases make only single-stranded DNA breaks and induce HDR without activating the mutagenic NHEJ pathway.

ZFNs are engineered double-strand break inducing agents comprised of a zinc finger DNA binding domain and a double strand break inducing agent domain. Engineered ZFNs consist of two zinc finger arrays (ZFAs), each of which is fused to a single subunit of a nonspecific endonuclease, such as the nuclease domain from the FokI enzyme, which becomes active upon dimerization. Typically, a single ZFA consists of 3 or 4 zinc finger domains, each of which is designed to recognize a specific nucleotide triplet (GGC, GAT, etc.). In certain embodiments, ZFNs composed of two “3-finger” ZFAs are capable of recognizing an 18 base pair target site; an 18 base pair recognition sequence is generally unique, even within large genomes such as those of humans and plants. By directing the co-localization and dimerization of two FokI nuclease monomers, ZFNs generate a functional site-specific endonuclease that creates a double-stranded break (DSB) in DNA at the targeted locus.

Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

CRISPR/Cas (CRISPR associated) (clustered regulatory interspaced short palindromic repeats) systems are loci that contain multiple short direct repeats, and provide acquired immunity to bacteria and archaea. CRISPR systems reply on crRNA and tracrRNA for sequence-specific silencing of invading foreign DNA. Three types of CRISPR systems exist: in type II systems, Cas9 serves as an RNA-guided DNA endonuclease that cleaves DNA upon crRNA-tracrRNA target recognition.

crRNA: CRISPR RNA base pairs with tracrRNA to form a two-RNA structure that guides the Cas9 endonuclease to complementary DNA sites for cleavage. A double-stranded break (DSB) is a form of DNA damage that occurs when both DNA strands are cleaved. DSBs can be products of TALENs, ZFNs, and CRISPR)/Cas9 action.

Homology-directed repair (HDR) is a template-dependent pathway for DSB repair. By supplying a homology-containing donor template along with a site-specific nuclease, HDR faithfully inserts the donor molecule at the targeted locus. This approach enables the insertion of single or multiple transgenes, as well as single nucleotide substitutions.

NHEJ (nonhomologous end joining) is a DSB repair pathway that ligates or joins two broken ends together. NHEJ does not use a homologous template for repair and thus typically leads to the introduction of small insertions and deletions at the site of the break.

PAMs (protospacer adjacent motifs) are short nucleotide motifs that occur on crRNA and are specifically recognized and required by Cas9 for DNA cleavage.

tracrRNA (transactivating chimeric RNA) is noncoding RNA that promotes crRNA processing and is required for activating RNA-guided cleavage by Cas9.

In one embodiment, the site-specific genome-editing method comprises contacting the host cell with one or more integration polynucleotides comprising an exogenous nucleic acid to be integrated into the genomic target site, and one or more nucleases capable of causing a double-strand break near or within the genomic target site. Cleavage near or within the genomic target site greatly increases the frequency of homologous recombination at or near the cleavage site.

In certain embodiments, a site-specific nuclease cleaves DNA in cellular chromatin, and facilitates targeted integration of an exogenous sequence (donor polynucleotide). In certain embodiments for targeted integration, one or more zinc finger or TALE DNA binding domains are engineered to bind a target site at or near the predetermined cleavage site, and a fusion protein comprising the engineered zinc finger or TALE DNA binding domain and a cleavage domain is expressed in a cell. Upon binding of the zinc finger or TALE DNA binding portion of the fusion protein to the target site, the DNA is cleaved, preferably via a double stranded break, near the target site by the cleavage domain. The presence of a double-stranded break facilitates integration of exogenous sequences as described herein via NHEJ mechanisms.

The exogenous (donor) sequence can be introduced into the cell prior to, concurrently with, or subsequent to, expression of the fusion protein(s).

“Recombination” refers to a process of exchange of genetic information between two polynucleotides. As used herein, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends:

A “cleavage domain” comprises one or more polypeptide sequences which catalytic activity for DNA cleavage.

A “cleavage half-domain” is a polypeptide sequence which, in conjunction with a second polypeptide (either identical or different), forms a complex having cleavage activity (preferably double-strand cleavage activity).

In one embodiment, the present invention employs markerless genomic integration of an exogenous nucleic acid using a site-specific nuclease. In one embodiment, an exogenous donor polynucleotide is introduced to a host cell, wherein the polynucleotide comprises a nucleic acid of interest (D) flanked by a first homology region (HR1) and a second homology region (HR2). HR1 and HR2 share homology with 5′ and 3′ regions, respectively, of a genomic target site (TS). A site-specific nuclease (N) is also introduced to the host cell, wherein the nuclease is capable of recognizing and cleaving a unique sequence within the target site. Upon induction of a double-stranded break within the target site by the site-specific nuclease, endogenous homologous recombination machinery integrates the nucleic acid of interest at the cleaved target site at a higher frequency as compared to a target site not comprising a double-stranded break.

Various methods are available to identify those cells having an altered genome at or near the target site without the use of a selectable marker. In some embodiments, such methods seek to detect any change in the target site, and include but are not limited to PCR methods, sequencing methods, nuclease digestion, e.g., restriction mapping, Southern blots, and any combination thereof.

Cleavage domains useful according to the present invention can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). Non limiting examples of homing endonucleases and meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

A recognition sequence is any polynucleotide sequence that is specifically recognized and/or bound by a double-strand break inducing agent. The length of the recognition site sequence can vary, and includes, for example, sequences that are at least 10, 12, 14, 16, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more nucleotides in length.

In some embodiments, the recognition sequence is palindromic, that is, the sequence on one strand reads the same in the opposite direction on the complementary strand. In some embodiments, the cleavage site is within the recognition sequence. In other embodiments, the cleavage site is outside of the recognition sequence. In some embodiments, cleavage produces blunt end termini. In other embodiments, cleavage produces single-stranded overhangs, i.e., “sticky ends,” which can be either 5′ overhangs, or 3′ overhangs.

In some embodiments of the methods provided herein, one or more of the nucleases is a site-specific recombinase. A site-specific recombinase, also referred to as a recombinase, is a polypeptide that catalyzes conservative site-specific recombination between its compatible recombination sites, and includes native polypeptides as well as derivatives, variants and/or fragments that retain activity, and native polynucleotides, derivatives, variants, and/or fragments that encode a recombinase that retains activity. The recognition sites range from about 30 nucleotide minimal sites to a few hundred nucleotides. Any recognition site for a recombinase can be used, including naturally occurring sites, and variants.

In some embodiments of the methods provided herein, one or more of the nucleases is a transposase. Transposases are polypeptides that mediate transposition of a transposon from one location in the genome to another. Transposases typically induce double strand breaks to excise the transposon, recognize subterminal repeats, and bring together the ends of the excised transposon. In some systems other proteins are also required to bring together the ends during transposition. Examples of transposons and transposases include, but are not limited to, the Ac/Ds, Dt/rdt, Mu-M1/Mn, and Spm(En)/dSpm elements from maize, the Tam elements from snapdragon, the Mu transposon from bacteriophage, bacterial transposons (Tn) and insertion sequences (IS), Ty elements of yeast (retrotransposon), Ta1 elements from Arabidopsis (retrotransposon), the P element transposon from Drosophila (Gloor, et al., (1991) Science 253:1110-1117), the Copia, Mariner and Minos elements from Drosophila, the Hermes elements from the housefly, the PiggyBack™ elements from Trichplusia ni, Tc1 elements from C. elegans, and IAP elements from mice (retrotransposon).

The Cre-LoxP recombination system is a site-specific recombination technology useful for performing site-specific deletions, insertions, translocations, and inversions in the DNA of cells or transgenic animals. The Cre recombinase protein (encoded by the locus originally named as “causes recombination”) consists of four subunits and two domains: a larger carboxyl (C-terminal) domain and a smaller amino (N-terminal) domain. The loxP (locus of X-over P1) is a site on the Bacteriophage P1 and consists of 34 bp. The results of Cre-recombinase-mediated recombination depend on the location and orientation of the loxP sites, which can be located cis or trans. In case of cis-localization, the orientation of the loxP sites can be the same or opposite. In case of trans-localization, the DNA strands involved can be linear or circular. The results of Cre recombinase-mediated recombination can be excision (when the loxP sites are in the same orientation) or inversion (when the loxP sites are in the opposite orientation) of an intervening sequence in case of cis loxP sites, or insertion of one DNA into another or translocation between two molecules (chromosomes) in case of trans loxP sites. The Cre-LoxP recombination system is known in the art, see, for example, Andras Nagy, Cre recombinase: the universal reagent for genome tailoring, Genesis 26:99-109 (2000).

The Lox-Stop-Lox (LSL) cassette prevents expression of the transgene in the absence of Cre-mediated recombination. In the presence of Cre recombinase, the LoxP sites recombine, and the stop cassette is deleted. The Lox-Stop-Lox (LSL) cassette is known in the art. See, Allen Institute for Brain Science, Mouse Brain Connectivity Altas, Technical White Paper: Transgenic Characterization Overview (2012).

### Materials for Practicing the Methods of the Subject Invention

The present invention also provides materials for replacing a dominantly acting nucleic acid sequence in animals. In one embodiment, the present invention provides a composition comprising a modification construct, a site-specific nuclease, and, optionally, one or more SSCs of a male animal whose genome contains a dominantly acting nucleic acid sequence.

Optionally, the composition may also comprise any material useful for performing the modification method of the present invention. The kit may also comprise, e.g., vectors, culture media, preservatives, diluents, components necessary for detecting the detectable agent (e.g., a selectable marker).

### Delivery Methods

The nucleic acids (including nucleic acid molecules encoding a site-specific nuclease or the correction construct) as described herein can be introduced into a cell using any suitable method. Nucleases can also be introduced directly into the cells. For example, two polynucleotides, each comprising sequences encoding one of the aforementioned polypeptides, can be introduced into a cell, and when the polypeptides are expressed and each binds to its target sequence, cleavage occurs at or near the target sequence. Alternatively, a single polynucleotide comprising sequences encoding both fusion polypeptides, is introduced into a cell. Polynucleotides can be DNA, RNA or any modified forms or analogues of DNA and/or RNA.

In certain embodiments, one or more proteins can be cloned into a vector for transfection of cells. Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties.

In certain embodiments, the nucleases and exogenous sequences are delivered in vivo or ex vivo in cells. Non-viral vector delivery systems for delivering polynucleotides to cells include DNA plasmids, naked nucleic acid, and nucleic acid complexed with a delivery vehicle such as a liposome or poloxamer.

Conventional viral based systems for the delivery of nucleases and nucleic acid molecules include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are a promising alternative gene delivery system based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Methods of non-viral delivery of nucleic acids in vivo or ex vivo include electroporation, lipofection, microinjection, biolistics, virosomes, liposomes (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787), immunoliposomes, polycation or lipid:nucleic acid conjugates, naked DNA, artificial virions, viral vector systems (e.g., retroviral, lentivirus, adenoviral, adeno-associated, vaccinia and herpes simplex virus vectors as described in WO 2007/014275 for delivering proteins comprising ZFPs) and agent-enhanced uptake of DNA.

Lipofection is described in for example, U.S. Pat. Nos. 5,049,386; 4,946,787; and 4,897,355 and lipofection reagents are sold commercially (e.g., Transfectam™. and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

Additional exemplary nucleic acid delivery systems include those provided by Amaxa Biosystems (Cologne, Germany), Maxcyte, Inc. (Rockville, Md.) and BTX Molecular Delivery Systems (Holliston, Mass.) and Copernicus Therapeutics Inc., (see for example U.S. Pat. No. 6,008,336).

Microinjection: Direct microinjection of DNA into various cells, including egg or embryo cells, has also been employed effectively for transforming many species. In the mouse, the existence of pluripotent embryonic stem (ES) cells that are culturable in vitro has been exploited to generate transformed mice. The ES cells can be transformed in culture, then micro-injected into mouse blastocysts, where they integrate into the developing embryo and ultimately generate germline chimeras. By interbreeding heterozygous siblings, homozygous animals carrying the desired gene can be obtained.

### Spermatogonial Stem Cell Transfer

Methods for performing spermatogonial stem cell transfer are known in the art.

In one embodiment, the SSC transfer method useful according to the present invention comprises:
providing spermatogonial stem cells (SSCs) from a male donor animal;
   introducing the donor SSCs into a reproductive organ of a sterile male recipient animal, whereby the sterile male recipient produces donor-derived, fertilization-competent, haploid male gametes; and optionally,
collecting the donor-derived, fertilization-competent, haploid male gametes produced by the sterile male recipient.

In certain embodiments, the SSC transfer method uses sterile, hybrid male recipient animals or sterile male recipient animals that have been genetically modified to have heritable male sterility.

In one embodiment, the recipient male animal is genetically modified such that it has an intact spermatogenic compartment but cannot perform spermatogenesis.

In certain embodiments, the sterile recipient animal can be produced via deletion or inactivating mutations of genes including, but not limited to, Deleted-in-Azoospermia like (DAZL); protamine genes (e.g., PRM1, PRM2) associated with DNA packaging in the sperm nucleus; genes in the azoospermia factor (AZF) region of the Y chromosome (such genes include, but are not limited to, USP9Y); and genes associated with male meiosis (such genes include, but are not limited to, HORMA domain-containing protein 1 (HORMAD1)). In another embodiment, the sterile recipient animal can be produced via genetic mutation(s) associated with sertoli cell-only syndrome (such genetic mutation includes mutations in USP9Y).

In one specific embodiment, the recipient male animal is genetically modified such that it does not express functional Deleted-in-Azoospermia like (DAZL) protein. In one specific embodiment, the recipient male animal is genetically modified such that the DAZL gene is deleted.

In one specific embodiment, the recipient male animal is genetically modified such that the DAZL gene does not encode functional DAZL protein.

As used herein, an inactivating mutation refers to any mutation (genetic alteration of a DNA molecule) that leads to an at least 30% reduction of function of the protein encoded by the DNA molecule. In one embodiment, the present invention provides a method for effecting spermatogonial stem cell (SSC) transfer, wherein the method comprises:
providing spermatogonial stem cells (SSCs) from a male donor animal;
introducing the donor SSCs into a reproductive organ of a sterile, hybrid male recipient animal, whereby the sterile, hybrid male recipient produces donor-derived, fertilization-competent, haploid male gametes; and optionally,
collecting the donor-derived, fertilization-competent, haploid male gametes produced by the sterile, hybrid male recipient.

The term “hybrid animal,” as used herein, refers to a crossbred animal with parentage of two different species. Hybrid male animals are usually sterile and cannot produce fertilization-competent, haploid male gametes. Examples of hybrid animals include, but are not limited to, mules (a cross between a horse and a donkey), ligers (a cross between a lion and a tiger), yattles (a cross between a yak and a buffalo), dzo (a cross between a yak and a bull), and hybrid animals that are crosses between servals and ocelots/domestic cats.

In another embodiment, the SSC transfer method useful according to the present invention comprises:
providing spermatogonial stem cells (SSCs) from a male donor animal;
introducing the donor SSCs into a reproductive organ of a genetically-modified, sterile male recipient animal, whereby the sterile male recipient produces donor-derived, fertilization-competent, haploid male gametes, and wherein the sterile male recipient animal is genetically modified such that it has an intact spermatogenic compartment but cannot perform spermatogenesis; and optionally,
collecting the donor-derived, fertilization-competent, haploid male gametes produced by the sterile male recipient.

In another embodiment, the present invention provides a method for effecting spermatogonial stem cell (SSC) transfer, wherein the method comprises:
providing spermatogonial stem cells (SSCs) from a male donor animal;
introducing the donor SSCs into a reproductive organ of a genetically-modified male recipient animal whereby the recipient produces donor-derived, fertilization-competent, haploid male gametes, wherein the recipient animal is genetically modified such that the native male gametes produced by the recipient animal express at least one detectable biomarker label; optionally,
distinguishing the native male gametes produced by the recipient animal from the donor-derived male gametes produced by the recipient animal based on the detectable biomarker label; and optionally,
collecting donor-derived, fertilization-competent, haploid male gametes produced by the recipient animal.

In one specific embodiment, the native male gametes produced by the recipient animal express at least one detectable cell surface biomarker (such as cell-surface antigen tag(s)).

In one embodiment, native male gametes produced by the recipient animal express luminescent proteins. In one embodiment, native male gametes produced by the recipient animal are distinguished from the donor-derived male gametes produced by the recipient animal by flow sorting, such as fluorescence activated cell sorting (FACS) and magnetic-activated cell sorting (MACS).

In one embodiment, the genetically-modified recipient male animal comprises a reporter gene for expression on the cell surface of native male gametes. In certain embodiments, the reporter gene encodes a luminescent protein.

The term “luminescent protein,” as used herein, refers to a protein that emits light. Luminescent proteins useful according to the present invention include, but are not limited to, fluorescent proteins including, but not limited to, green fluorescent protein, yellow fluorescent protein, cyan fluorescent protein, and red fluorescent protein; and phosphorescent proteins. Fluorescent proteins are members of a class of proteins that share the unique property of being self-sufficient to form a visible wavelength chromophore from a sequence of three amino acids within their own polypeptide sequence. A variety of luminescent proteins, including fluorescent proteins, are publicly known. Fluorescent proteins useful according to the present invention include, but are not limited to, the fluorescent proteins disclosed in U.S. Pat. No. 7,160,698, U. S. Application Publication Nos. 2009/0221799, 2009/0092960, 2007/0204355, 2007/0122851, 2006/0183133, 2005/0048609, 2012/0238726, 2012/0034643, 2011/0269945, 2011/0223636, 2011/0152502, 2011/0126305, 2011/0099646, 2010/0286370, 2010/0233726, 2010/0184116, 2010/0087006, 2010/0035287, 2007/0021598, 2005/0244921, 2005/0221338, 2004/0146972, and 2001/0003650, all of which are hereby incorporated by reference in their entireties.

In one embodiment, donor SSCs are introduced into the testis of the male recipient animal.

In one embodiment, male gametes produced by the recipient animal are sperm.

In one embodiment, the donor spermatogonial stem cells (SSCs) embody a genetic background of interest. In one specific embodiment, the donor animal is from the Genus of Bos, including but not limited to, Bos Taurus (domestic cattle).

In certain embodiments, the recipient animal can be adult animals or immature animals. In one embodiment, the recipient animal is in puberty.

In a further embodiment, the present invention further comprises the step of fertilizing an egg from an animal species of interest with the donor-derived, fertilization-competent, haploid male gamete produced by the recipient animal. Methods of fertilization of eggs are known in the art, and include, but are not limited to, intracytoplasmic sperm injection (ICSI) and round spermatid injection (ROSI).

Parentages of the recipient hybrid animal, the recipient animal, and/or the donor animal can be of any animal species including, but not limited to, species of cats; mice; rats; wolves; coyotes; dogs; chinchillas; deer; muskrats; lions; tigers; pigs; hamsters; horses; cattle; sheep; goats; ducks; geese; chickens; primates such as apes, chimpanzees, orangutans, monkeys; and humans.

In certain embodiments, one or both parentages of the recipient hybrid animal, the recipient animal, and/or the donor animal can be of any vertebrates, including fish, amphibians, birds, and mammals. In certain embodiments, one or both parentages of the recipient hybrid animal, the recipient animal, and/or the donor animal are not a human.

In certain embodiments, one or both parentages of the recipient hybrid animal, the recipient animal, and/or the donor animal can be from any family of Equidae, Bovidae, Canidae, Felidae, and Suidae.

Mammalian spermatogonial stem cells (SSCs) self-renew and produce daughter cells that commit to differentiate into spermatozoa throughout adult life of the male. SSCs can be identified by functional assays known in the art, such as transplantation techniques in which donor testis cells are injected into the seminiferous tubules of a sterile recipient.

In one embodiment, donor spermatogonial stem cells can be cryopreserved and/or cultured in vitro. Frozen spermatogonial stem cells can be grown in vitro and cryopreserved again during the preservation period.

SSCs can be cultured in serum-containing or serum-free medium. In one embodiment, the cell culture medium comprises Dulbecco's Modified Eagle Medium (DMEM), and optionally, fetal calf serum.

In certain embodiments, SSC culture medium can comprise one or more ingredients including, but not limited to, glial cell-derived neurotrophic factor (GDNF), fibroblast growth factor-2 (FGF2), leukemia inhibitory factor (LIF), insulin-like growth factor-I (IGF-I), epidermal growth factor (EGF), stem cell factor (SCF), B27-minus vitamin A, Ham's F12 nutrient mixture, 2-mercaptoethanol, and L-glutamine.

Methods for transplanting spermatogonial stem cells into recipient reproductive organs (such as, the testis) are known in the art. Transplantation can be performed by direct injection into seminiferous tubules through microinjection or by injection into efferent ducts through microinjection, thereby allowing SSCs to reach the rete testis of the recipient. The transplanted spermatogonial stem cells adhere to the tube wall of the recipient seminiferous tubules, and then differentiate and develop into spermatocytes, spermatids and spermatozoa, and finally mature following transfer to the epididymis.

Methods for the introduction of one or more SSCs into a recipient male also include injection into the vas deferens and epididymis or manipulations on fetal or juvenile testes, techniques to sever the seminiferous tubules inside the testicular covering, with minimal trauma, which allow injected cells to enter the cut ends of the tubules. Alternatively, neonatal testis (or testes), which are still undergoing development, can be used.

### EXAMPLES

Following are examples that illustrate procedures and embodiments for practicing the invention. These examples should not be construed as limiting.

### Example 1

In one embodiment, the present invention provides a method for making a recessive gene dominant using polycistronic miRNA-based suppression of the dominant version combined with an expression construct carrying a miRNA-resistant version of the previously recessive gene thus making such gene dominant.

Coat color in animals is brought about by a single pigment, melanin. There are two classes of melanin: pheomelanin, which produces a blond or red color, and eumelanin, which produces a dark brown or black color. Both classes of melanin are synthesized from tyrosine, but their synthetic pathways diverge after production of dopaquinone. The primary switch controlling whether a particular melanocyte produces pheomelanin or eumelanin is the melanocortin receptor.

In Black Angus cattle, the black coat color is caused by an activating mutation in the melanocortin receptor. In these cattle, the melanocortin receptor is always “on” resulting in the dominant trait of black coat color. In accordance with the present invention, the Black Angus version of the melanocortin receptor is knocked out using the polycistronic miRNA, and then a functional copy of the melanocortin receptor, that has been modified to make it no longer match the miRNA, is added in.

### Example 2

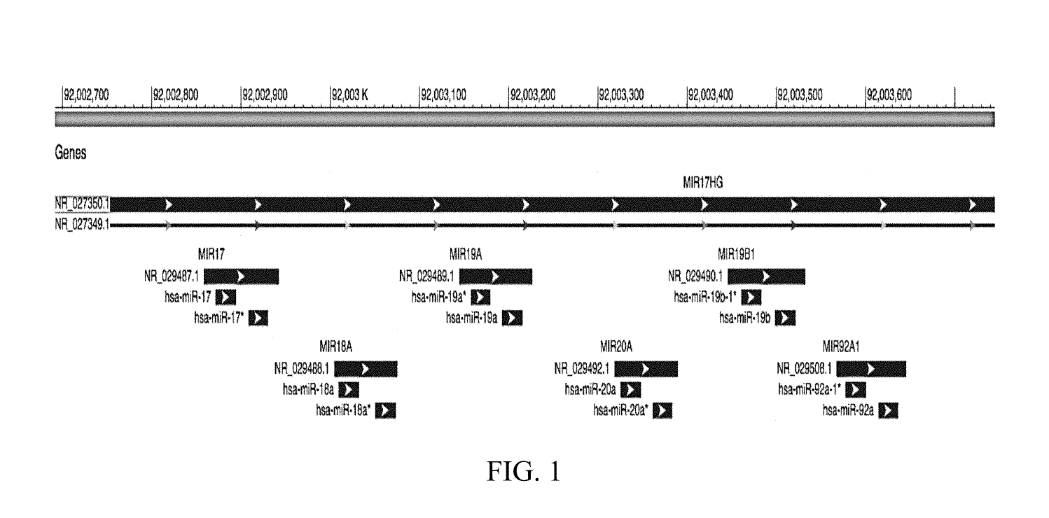

SEQ ID NO:1 and FIG. 1 show a plasmid vector for a construct for use according to the method of the subject invention. The 13512 bp segment between NotI and BglII is the portion of the sequence that would be integrated into the animal's genome through any of a variety of methods.

This construct contains several important features.

(1) A tissue-specific promoter (in this case, the tyrosinase promoter, but that would change depending on the desired location of expression);

(2) A mutant form of PMEL to produce white coat coloration. In addition to the mutation, the sequence has been altered so that it is no longer inhibited by the miRNA;

(3) Four different small interfering RNAs, incorporated into miR30 flanking regions and loop. Note that, in order to avoid secondary structure, the miR30 sequences from four different species have been used (in this case, human, mouse, dog, and nelore cattle). Any sufficiently different miR30 could be used; however, although they fall into groups; the human miR30 is nearly identical to that from rhesus monkeys, chimps, and gorillas. The mouse sequence is nearly identical to that from rats. The dog sequence is nearly identical to that from bears and giant pandas. The nelore cattle is nearly identical to that in other cattle breeds and in sheep. Finally, while a set of four miR30 is specifically exemplified here, any native string of miR could be used. For example, the 17-92 cluster, or the 25-93-106 cluster, could have the miR sequences replaced, retaining the 5′,3′, and loop structures;

(4) A polyadenylation sequence, intron, and enhancer. SV40 is used here, but any of a great number are usable.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures, tables, and sequences, to the extent they are not inconsistent with the explicit teachings of this specification.

1: A method for enabling expression of a first allele of a gene over an endogenous second allele of the gene in an animal cell comprising at least one of:
a) increasing expression of the first allele of the gene; and
b) decreasing expression of the endogenous second allele of the gene by expressing an exogenous inhibitory RNA nucleic acid in the cell that binds to the mRNA transcribed from the endogenous second allele of the gene.

2: The method, according to claim 1, wherein step a) comprises introducing an exogenous polynucleotide sequence encoding the first allele of the gene into the cell.

3: The method, according to claim 1, wherein step a) comprises changing the polynucleotide sequence of the first allele of the gene by genome editing the cell such that the exogenous inhibitory RNA nucleic acid that would normally inhibit the expression of the first allele of the gene no longer binds to the mRNA transcribed from the first allele of the gene.

4: The method, according to claim 3, wherein the change to the polynucleotide sequence of the first allele of the gene does not result in a change to the amino acid sequence of the encoded protein or, if there is a change, it does not adversely affect the functionality of the protein.

5: The method, according to claim 4, wherein one or more changes are made based on the degeneracy of the genetic code.

6: The method, according to claim 2, wherein the exogenous polynucleotide sequence is from a gene encoding the same protein in a second species.

7. (canceled)

8: The method, according to claim 1, wherein multiple exogenous inhibitory RNA nucleic acids are expressed that bind to the mRNA transcribed from the endogenous second allele of the gene at the 3′ untranslated region (UTR).

9: The method, according to claim 8, wherein the multiple exogenous inhibitory RNA nucleic acids that target the endogenous second allele of the gene are provided in polycistronic strings.

10: The method, according to claim 1, utilizing somatic cell nuclear transfer (SCNT).

11: The method, according to claim 10, wherein the somatic cell is a skin fibroblast.

12: The method of claim 1, wherein the animal cell is a spermatogonial stem cell (SSC) and steps a) and b) comprise:
obtaining one or more spermatogonial stem cells (SSCs) of a male animal that has a dominantly acting endogenous nucleic acid molecule;
providing a modification construct comprising an exogenous polycistronic inhibitory RNA nucleic acid sequence comprising the exogenous inhibitory RNA nucleic acid, and further providing an exogenous nucleic acid sequence comprising the first allele of the gene in which a base mutation in at least one codon has been introduced or exists (compared to the wild-type sequence of the recessively acting nucleic acid molecule in that species) such that binding of the exogenous inhibitory RNA nucleic acid is prevented or reduced; and
introducing the modification construct(s) into the SSC, thereby obtaining an SSC comprising a nucleic acid molecule that decreases expression of the endogenous second allele of the gene and the exogenous nucleic acid sequence; and
introducing the modified SSC into a reproductive organ of a male recipient animal; and optionally,
collecting the donor-derived, fertilization-competent, haploid male gametes produced by the male recipient.

13: The method, according to claim 1, wherein step a) comprises introducing into the cell an exogenous nucleic acid molecule, operably linked to a promoter, wherein the exogenous nucleic acid comprises the first allele of the gene, except that the nucleic acid sequence of the exogenous molecule differs from the naturally-occurring sequence such that interaction with the exogenous inhibitory RNA nucleic acid is reduced.

14: The method, according to claim 13, wherein said exogenous nucleic acid molecule encodes a protein encoded by a naturally-occurring recessively acting nucleic acid sequence, except that the nucleic acid sequence of the exogenous molecule differs from the naturally-occurring sequence such that interaction with endogenous inhibitory RNA molecules is reduced.

15: The method, according to claim 1, step b) comprises introducing into the cell an exogenous, polycistronic inhibitory RNA coding sequence comprising the exogenous inhibitory RNA nucleic acid, operably linked to a promoter, wherein the exogenous inhibitory RNA coding sequence encodes multiple inhibitory RNA molecules that decrease the expression of the endogenous second allele of the gene in the animal cell.

16: A non-human transgenic animal cell comprising:
a dominantly acting endogenous nucleic acid molecule encoding a protein and a recessively acting endogenous nucleic acid molecule;
an exogenous, polycistronic inhibitory RNA coding sequence, operably linked to a promoter, wherein the exogenous inhibitory RNA coding sequence encodes multiple inhibitory RNA molecules that decrease the expression of the dominantly acting endogenous nucleic acid molecule of the animal; and/or
an exogenous nucleic acid molecule, operably linked to a promoter, wherein the exogenous nucleic acid sequence encodes a protein encoded by the naturally-occurring recessively acting nucleic acid sequence, except that the nucleic acid sequence of the exogenous molecule differs from the naturally-occurring sequence such that interaction with endogenous inhibitory RNA molecules is reduced.

17: The cell of claim 16, wherein said cell comprises an exogenous, polycistronic inhibitory RNA coding sequence, operably linked to a promoter, wherein the exogenous inhibitory RNA coding sequence encodes multiple inhibitory RNA molecules that decrease the expression of the dominantly acting endogenous nucleic acid molecule of the animal.

18: The cell of claim 16, wherein said cell comprises an exogenous nucleic acid molecule, operably linked to a promoter, wherein the exogenous nucleic acid sequence encodes a protein encoded by a naturally-occurring recessively acting nucleic acid sequence, except that the nucleic acid sequence of the exogenous molecule differs from the naturally-occurring sequence such that interaction with endogenous inhibitory RNA molecules is reduced.

19: The cell of claim 16, wherein said cell comprises both an exogenous, polycistronic inhibitory RNA coding sequence, operably linked to a promoter, wherein the exogenous inhibitory RNA coding sequence encodes multiple inhibitory RNA molecules that decrease the expression of the dominantly acting endogenous nucleic acid molecule of the animal; and an exogenous nucleic acid molecule, operably linked to a promoter, wherein the exogenous nucleic acid sequence encodes a protein encoded by a naturally-occurring recessively acting nucleic acid sequence, except that the nucleic acid sequence of the exogenous molecule differs from the naturally-occurring sequence such that interaction with endogenous inhibitory RNA molecules is reduced.

20: The cell according to claim 16, wherein the cell is of a bovine.

21: The method of claim 3, wherein the exogenous polynucleotide sequence was altered such that the exogenous inhibitory RNA nucleic acid that would otherwise inhibit the expression of the recessive allele of the gene does not bind to the mRNA transcribed from the exogenous polynucleotide sequence.
